(19) United States
(12) Patent Application Publication
(10) Pub. No.: US 2021/0353877 A1
(43) Pub. Date: Nov. 18, 2021

(54) DRUG DELIVERY DEVICE

(71) Applicant: Sanofi-Aventis Deutschland GMBH, Frankfurt am Main (DE)

(72) Inventors: Michael Schabbach, Frankfurt am Main (DE); Beate Franke, Frankfurt am Main (DE); Giuliano Pradel, Massanzago (PD) (IT); Andrew Nelson, Dallas, TX (US)

(21) Appl. No.: 16/614,231

(22) PCT Filed: May 16, 2018
(86) PCT No.: PCT/EP2018/062814

§ 371 Date: Nov. 15, 2019

(30) Foreign Application Priority Data
May 18, 2017 (EP) 17171738.2

(51) Int. Cl. A61M 5/32; A61M 5/142

(52) U.S. Cl. CPC A61M 5/3245; A61M 5/14248; A61M 2005/14268; A61M 2005/3247; A61M 2005/14256

(57) ABSTRACT

The present disclosure relates to a drug delivery device. The drug delivery device includes a housing that has a surface adapted to be placed against a skin of a user during use of drug delivery device, and an attachment mechanism for holding the drug delivery device on the skin of the user. The drug delivery device also has a needle assembly, which includes a needle that protrudes from the surface for delivery of the drug to the user and a shield that surrounds the needle after the drug delivery device has been used. The needle assembly can be detached from the housing for disposal.

### CROSS-REFERENCE TO RELATED APPLICATION

The present application is the national stage entry of International Patent Application No. PCT/EP2018/062814, filed May 16, 2018, and claims priority to Application No. EP 17171738.2, filed on May 18, 2017, the disclosures of which are incorporated herein by reference.

### TECHNICAL FIELD

The present disclosure relates to a drug delivery device that includes a needle.

### BACKGROUND

Drug delivery devices such as large volume devices (“LVDs”) or patch pumps, typically have a needle for piercing a user's skin and delivering a medicament. After use, it is necessary to dispose of at least a part of the drug delivery device, particularly the needle, in an appropriate manner, for example in a ‘sharps bin’.

### SUMMARY

Some aspects of the present disclosure provide an advantageous drug delivery device that facilitates disposal of the needle after use of the drug delivery device.

Some aspects of the present disclosure provide a drug delivery device comprising: a housing having a surface adapted to be placed against a skin of a user during use of drug delivery device; an attachment mechanism for holding the drug delivery device on the skin of the user; and a needle assembly having a needle that protrudes from the surface for delivery of a drug, and a shield that surrounds the needle after use of the drug delivery device; and wherein the needle assembly is detachable from the housing for disposal.

The attachment mechanism may be attached to the housing, for example the surface of the housing, and is adapted to hold the drug delivery device on said user's skin.

The needle may be movable between a retracted position and an extended position in which the needle protrudes from the surface.

The needle may be movably mounted to the shield. For example, the needle may be slidably mounted to the shield.

The drug delivery device may further comprise a needle actuation mechanism adapted to move the needle from the retracted position to the extended position.

The shield may be moveable between a retracted position and an extended position. The shield and the needle may be arranged to move between the retracted position and the extended position independently of each other.

The shield may be adapted to move from the retracted position to the extended position after use of the drug delivery device.

In some examples, the drug delivery device further comprises a biasing member arranged to urge the shield towards the extended position.

The drug delivery device may further comprise a latch adapted to hold the shield in the retracted position prior to use of the drug delivery device.

In one example, the attachment mechanism includes an adhesive to adhere the surface of the housing to the user's skin.

In one example, the surface of the housing includes a recess in which the shield and optionally also the needle are disposed prior to use. Before use, the needle and shield are in a retracted position within the recess. During use the needle and shield move into an extended position where they protrude from the surface.

In some examples, the shield may comprise a groove and the housing may comprise a lug that can move within the groove to control movement of the shield relative to the housing.

The groove may comprise a first portion that defines movement of the shield from the retracted position to the extended position; and, a second portion that allows the shield to be detached from the housing.

In other examples, the needle assembly and housing may be threadingly attached.

The drug delivery device may further comprise a locking mechanism arranged to lock the needle to the shield. The locking mechanism may be engaged after use of the device, for example when both the shield and the needle are in an extended position.

The drug delivery device may be arranged such that a replacement needle assembly may be connected to the drug delivery device after removal of a needle assembly. In that way, the housing and other features of the drug delivery device can be reused and the needle and shield can be replaced.

The drug delivery device may further comprise a reservoir for holding a medicament. A fluid connector may be provided between the reservoir and the needle for carrying medicament from the reservoir to the needle. The fluid connector may be flexible and/or extendable.

In some examples, the reservoir comprises a plunger that is moved into the reservoir to displace the medicament therefrom. The plunger may move in a direction perpendicular to the longitudinal axis of the needle. In other words, the reservoir may be arranged such that that plunger moves in a direction parallel to the surface of the housing that is placed against a user's skin during use.

The drug delivery device may further comprise a reservoir that contains a medicament.

According to a further aspect of the present disclosure, there is provided a method of using a drug delivery device, the drug delivery device comprising a housing, and a needle assembly having a needle and a shield, wherein the method comprises: using an attachment mechanism to hold the drug delivery device on the skin of the user; delivering a drug to the user via the needle; moving the shield into a position in which it surrounds the needle after use of the drug delivery device; and, detaching the needle assembly from the housing.

These and other aspects of the disclosure will be apparent from and elucidated with reference to the embodiments described hereinafter.

### BRIEF DESCRIPTION OF THE DRAWINGS

Embodiments of the invention will now be described, by way of example only, with reference to the accompanying drawings, in which:

FIG. 1A is a schematic view of an drug delivery device, with the needle and shield in retracted positions;

FIG. 1B is a schematic view of the drug delivery device of FIG. 1A, with the needle in an extended position and the shield in a retracted position;

FIG. 1C is a schematic view of the drug delivery device of FIG. 1A and FIG. 1B, with the needle and shield in an extended position;

FIG. 1D is a schematic view of the drug delivery device of FIGS. 1A to 1C, with the needle assembly detached;

FIG. 2A is a schematic view of an drug delivery device, with a spring-loaded shield;

FIG. 2B is a schematic view of the drug delivery device of FIG. 2A, with the shield in a retracted position during use of the drug delivery device;

FIG. 2C is a schematic view of the drug delivery device of FIG. 2A and FIG. 2B, with the needle assembly detached;

FIG. 3A is a schematic view of a needle assembly and housing of the drug delivery devices of FIGS. 1A to 2C, with the shield in a retracted position;

FIG. 3B is a schematic view of the needle assembly and housing of FIG. 3A, with the shield in an extended position;

FIG. 3C is a schematic view of the needle assembly and housing of FIG. 3A and FIG. 3B, during removal of the needle assembly from the housing;

FIG. 3D is a schematic view of the needle assembly and housing of FIGS. 3A to 3C, the needle assembly having been detached from the housing; and,

FIG. 4 is a schematic view of an alternative needle assembly and housing of the drug delivery devices of FIGS. 1A to 2C, with the shield in a retracted position.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician. The device can include a cartridge-based system that requires piercing a sealed ampule before use. Volumes of medicament delivered with these various devices can range from about 0.5 ml to about 2 ml. In some examples, the device can include a large volume device (“LVD”) or patch pump, configured to be held on a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 17 and 29 Gauge.

The drug delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of a drug delivery device may each be activated via an activation mechanism. Such an activation mechanism can include an actuator, for example, one or more of a button, a lever, or other activation component. Activation of an automated function may be a one-step or multi-step process. That is, a user may need to activate one or more activation components in order to cause the automated function.

In addition, activation of one automated function may activate one or more subsequent automated functions, thereby forming an activation sequence. For example, activation of a first automated function may activate at least two of needle insertion, medicament injection, and needle retraction. Some drug delivery devices may also require a specific sequence of steps to cause the one or more automated functions to occur. Other devices may operate with a sequence of independent steps.

Some drug delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

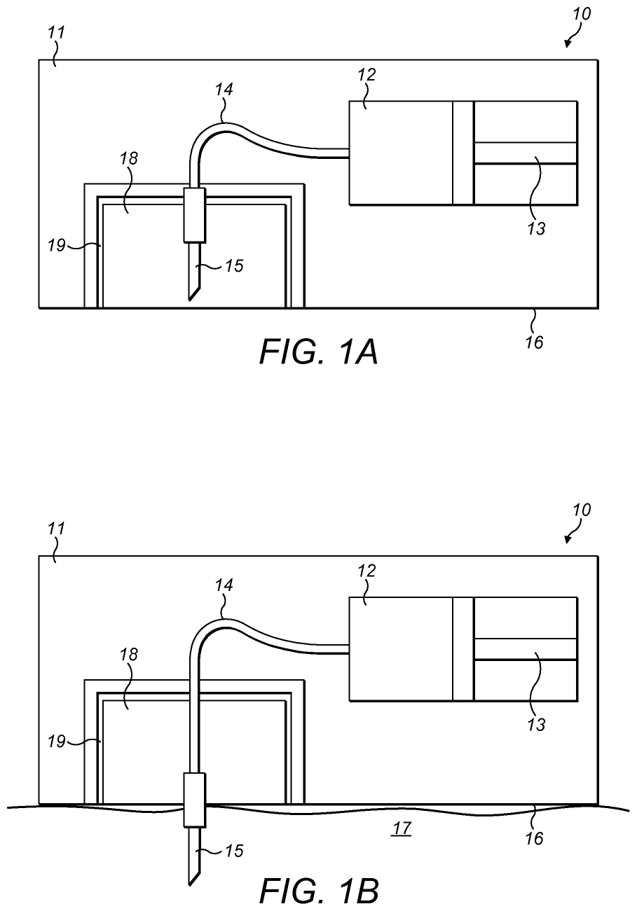

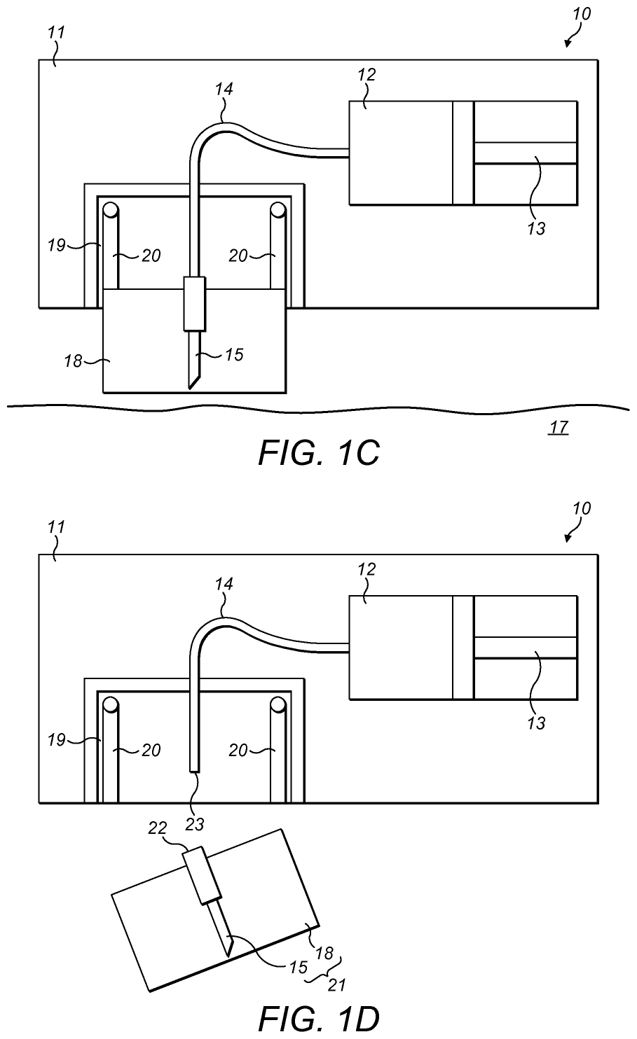

According to some embodiments of the present disclosure, an exemplary drug delivery device 10 is shown in FIGS. 1A, 1B, 1C and 1D. Drug delivery device 10, as described above, is configured to inject a medicament into a patient's body. Drug delivery device 10 includes a housing 11 which typically contains a reservoir 12 containing the medicament to be injected (e.g., a syringe) and the components required to facilitate one or more steps of the delivery process.

In this example, a plunger 13 is provided to push medicament from the reservoir 12 into a pipe 14. The end of the pipe 14 is connected to a needle 15 that delivers the medicament to the user. However, it will be appreciated that alternative manual or automatic drug delivery mechanisms may be provided instead of, or in addition to, the plunger 33.

As shown in FIG. 1A, the reservoir 12 is arranged perpendicularly to the needle 15. That is, during use the plunger 13 moves in a direction substantially perpendicular to the longitudinal direction of the needle 15. In other words, the plunger 13 moves in a direction substantially parallel to the skin 17 of the user during use (see FIG. 1B). In this way, the height of the drug delivery device 10 can be limited.

During use, a bottom surface 16 of the housing 11 is held against the skin 17 of the user. This may include use of an attachment mechanism to attach the drug delivery device 10 to the skin 17 of the user. In one example, the bottom surface 16 includes adhesive to adhere the drug delivery device 10 to the skin 17 of the user. In another example, the housing 11 may include loops to which a strap is attached, the strap being used to hold the drug delivery device 10 in place against the skin 17 of the user. However, other attachment mechanisms may be used to hold the drug delivery device 10 against the skin 17 of the user.

As shown in FIG. 1A, in this example the needle 15 is initially in a retracted position. In the retracted position the needle 15 is located entirely within the housing 11 and does not extend past the plane of a bottom surface 16 of the housing 11 and so cannot be accessed or accidentally pierce the skin 17 of a user.

In FIG. 1B the needle 15 has moved into an extended position. In the extended position the needle 15 pierces skin 17 of the user to deliver a medicament. The needle 15 may move into the extended position prior to the drug delivery device 10 being placed against the skin 17 of the user, or it may move after the drug delivery device 10 has been placed against the skin 17 of the user. Movement of the needle 15 can be performed by a manual or automated needle insertion mechanism.

Movement of the needle 15 from the retracted to the extended position can occur via several mechanisms. For example, the drug delivery device 10 may include an actuator, such as a button or lever, that pushes the needle 15 into the extended position when actuated by the user.

Alternatively, movement of the needle 15 may be “automated”, whereby the needle 15 moves relative to the housing 11 and can be triggered by movement of an actuator, such as a button or lever, or the automated movement is triggered by placing the drug delivery device 10 against the skin 17 of the user. In one example, an actuator may be moved relative to the drug delivery device 10 on placing the drug delivery device 10 against a skin 17 of the user, triggering the automated movement of the needle 15. The automated movement may be driven by a biasing member, for example a spring that pushes the needle 15 into the extended position. A latch may be provided to hold the spring and needle 15 in a pre-loaded position, and the actuator may release the latch so that the spring can push the needle 15 into the extended position shown in FIG. 1B.

A lock may be provided to hold the needle 15 in the extended position, preventing it from moving back to the retracted position.

Other manual or automated features can be included with the medicament delivery mechanism for drug injection. Injection is the process by which the plunger 13 is moved into the reservoir 12 in order to force a medicament into the pipe 14 and the needle 15. In some embodiments, a drive spring (not shown) is under compression before drug delivery device 10 is activated. A latch may hold the drive spring and plunger 13 in a pre-loaded position, and an actuator may be provided to release the latch and begin delivery of the medicament. The latch and actuator may be the same latch and actuator that affect movement of the needle 15 into the extended position, as described above. In other embodiments, a manual actuator, such as a button or lever, is provided for the user to push the plunger 13 into the reservoir 12 and push medicament into the needle 15.

As illustrated in FIG. 1A and FIG. 1B, the pipe 14 that connects the reservoir 12 to the needle 15 is flexible and/or extendable, so that the fluid connection between the reservoir 12 and needle 15 is not affected by the movement of the needle 15 relative to the reservoir, as the needle 15 moves into the extended position.

The drug delivery device 10 is in the condition shown in FIG. 1B during the injector process, that is, until the appropriate amount of medicament has been injected. After use, the drug delivery device 10 is removed from the skin 17 of the user, as shown in FIG. 1C.

As shown in FIG. 1C, on removal of the drug delivery device 10 from the skin a shield 18 extends from the housing 11 to surround the needle 15.

The shield 18 has moved from a retracted position, shown in FIG. 1A and FIG. 1B, into an extended position, shown in FIG. 1C. In the retracted position the shield 18 is within a recess 19 in the housing 11 and the needle 15 can extend past the shield 18 (and housing 11) for use. After use, the shield 18 is moved to an extended position to protect the needle 15 and also protect the user and others from being pierced by the needle 15 after the drug delivery device 10 has been used. In the retracted position the shield 18 is received in a recess 19 in the housing 11.

In this example, the shield 18 has a generally cylindrical shape and surrounds the needle 15, with the needle 15 being located in the hollow interior of the shield 18. However, in alternative examples the shield 18 may have an alternative tubular shape, for example square, rectangular, or hexagonal with the needle 15 being located within the shield 18. Alternatively, the shield 18 may comprise a wall that abuts against the needle 15 without surrounding the needle 15.

As shown in FIG. 1C, in this example the shield 18 is slidably mounted to the housing 11 on guides 20, which allow the shield 18 to slide from the retracted position into the extended position. The shield 18 may include engaging members that interact with the guides 20 to permit the sliding movement. Alternatively, the guides 20 may comprise engaging members that interact with the shield 18 to permit the sliding movement. The guides 20 may comprise a groove, a protrusion, a linear bearing, or other feature that permits movement of the shield 18. The guides 20 may be omitted if the shield 38 and recess 39 are shaped correspondingly so that the shield 38 slides into and out of the recess 39.

As explained previously, movement of the shield 18 from the retracted position to the extended position may be manually or automatically actuated.

If movement of the shield 18 is manually actuated, then the drug delivery device 10 may include an actuator, for example a button or lever, which the user can use to move the shield 18 from the retracted position to the extended position after using the device.

If movement of the shield 18 is automated, energy for the automated movement of the shield 18 can be provided by one or more energy sources. Energy sources can include, for example, mechanical, pneumatic, chemical, or electrical energy. For example, mechanical energy sources can include springs, levers, elastomers, or other mechanical mechanisms to store or release energy. The drug delivery device 10 may include one or more energy sources. The drug delivery device 10 can further include gears, valves, or other mechanisms to convert energy into movement of the shield 18 or other components of the drug delivery device 10.

The movement of the shield 18 from the retracted position to the extended position may be activated via an activation mechanism. Such an activation mechanism can include an actuator, for example, one or more of a button, a lever, or other activation component. Activation of the movement of the shield 18 may be a one-step or multi-step process. That is, a user may need to activate one or more activation components in order to cause the automated movement of the shield 18.

In addition, movement of the shield 18 may activate one or more subsequent automated functions, thereby forming an activation sequence. For example, movement of the shield 18 from the retracted position to the extended position may activate the end of the movement of the plunger 13, switching off of the drug delivery device 10, or other automated function. In another example, the end of the movement of the plunger 13 may activate movement of the shield 18 from the retracted position to the extended position. Alternatively, automatic movement of the shield 18 may be activated by a timer, a sensor, an actuator that engages with the skin, or other function.

The drug delivery device 10 may also require a specific sequence of steps to cause the one or more automated functions to occur. The drug delivery device 10 may operate with a sequence of independent steps.

As shown in FIG. 1D, after use, once the shield 18 has moved into the extended position, the shield 18 and the needle 15 can be removed from the housing 11. The shield 18 and the needle 15 together form a needle assembly 21 that can be detached from the housing 11 and disposed of. By detaching the shield 18 and needle 15 together the detached needle assembly 21 does not have any protruding needle, making it safer to remove the needle 15 from the housing 11 and also to dispose of it.

The needle 15 may be movably connected to the shield 18. For example, the needle 15 may include a protrusion that is received in a groove within the shield 18. Alternatively, the shield 18 may include a tube in which the needle 15 is received, allowing the needle 15 to slide within the tube. In this way, the needle 15 can move from the retracted to the extended position while the shield 18 stays stationary (as illustrated in FIG. 1B), and the shield 18 can move from the retracted to the extended position while the needle 15 stays stationary (as shown in FIG. 1C). However, once the needle assembly 21 is removed from the housing 11 the protrusion and groove hold the needle 15 and shield 18 together. A lock may be provided to hold the needle 15 in position relative to the shield 18 after the shield 18 moves into the extended position.

As shown in FIG. 1D, on removal of the needle assembly 21 from the housing 11 the pipe 14 is disconnected from the needle 15. To allow this, the pipe 14 is detachably connected to the needle 15. For example, an end 22 of the needle 15 is received in the end 23 of the pipe, and the pipe 14 can be pulled off the end 22 of the needle 15. The end 22 of the needle 15 may include a bulbous section that is received in the end 23 of the pipe 14, to increase the holding force between the pipe 14 and the needle 15 but still allow detachment. Alternatively, the end 23 of the pipe 14 may be received in the end 22 of the needle 15.

As explained above, the needle assembly 21 (including shield 18 and needle 15) is detachable from the housing 11.

In one example, the needle assembly 21 may be threadingly attached to the housing 11, with the recess 19 of the housing 11 comprising a female thread and the shield 18 comprising a male thread (or vice versa). In this way, twisting the shield 18 relative to the housing 11 will unscrew the needle assembly 21 and allow the needle assembly 21 to be pulled away from the housing 11, which in turn disconnects the pipe 14 from the needle 15.

In another example, the needle assembly 21 is attached to the housing 11 by a bayonet fitting, allowing the needle assembly 21 to be detached from the housing 11. In this example, the recess 19 of the housing 11 may include one or more lugs that engage with a bayonet slot on the shield 18 (or vice versa).

In another example, the needle assembly 21 is attached to the housing 11 by a combination of a thread attachment and a bayonet attachment.

In another example, the needle assembly 21 is attached to the housing 11 by a push-fit, where the shield 18 is pushed into the recess 19 and held by friction or by some part of the recess 19 and/or shield 18 deforming under pressure. Deformable holding tabs may be provided on the housing 11 and/or shield 18 for this purpose. Such a push-fit allows the needle assembly 21 to be detached from the housing 11 by pulling the shield 18 out of the recess 19.

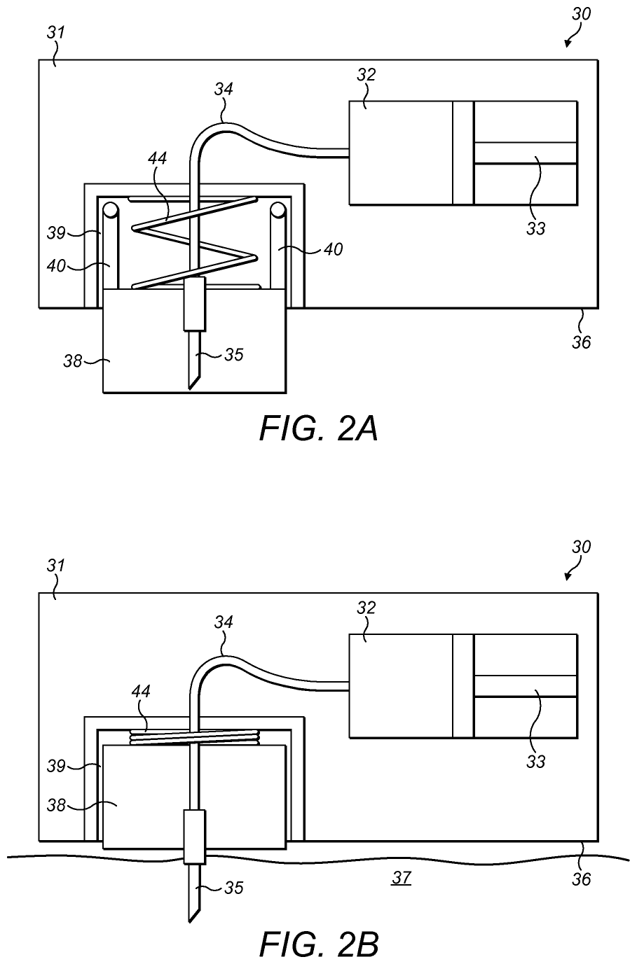

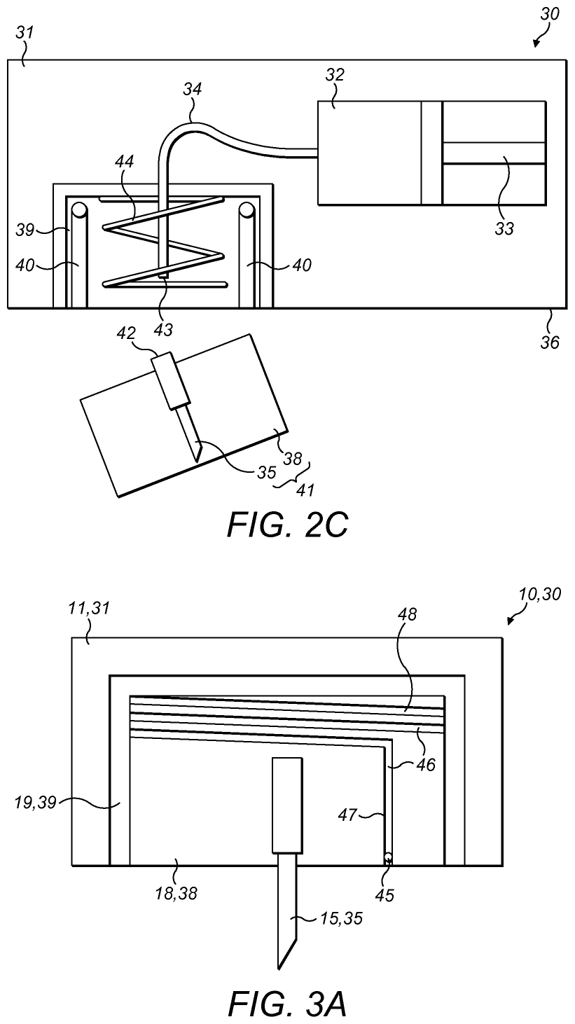

According to some embodiments of the present disclosure, a further drug delivery device 30 is shown in FIGS. 2A, 2B and 2C. The drug delivery device 30 is similar to the embodiments of FIG. 1A to 1D, and is configured to inject a medicament into a patient's body. The device includes a housing 31 which typically contains a reservoir 32 containing the medicament to be injected (e.g., a syringe) and the components required to facilitate one or more steps of the delivery process, for example a plunger 33.

The reservoir 32, plunger 33, pipe 34, and plunger 33 are as substantially as described above with reference to FIG. 1A to 1D.

However, in this embodiment, the needle 35 does not move between a retracted position and an extended position. In this embodiment the needle 35 is in a fixed position and extends beyond the plane of a bottom surface 36 of the housing 31. As the needle 35 of this embodiment does not move, there is no need for the pipe 34 to be flexible or extendable. However, the pipe 34 may be flexible and/or extendable.

The embodiment of FIG. 2A to 2C has a spring-loaded shield 38. FIG. 2A shows the drug delivery device 30 prior to use, and in this position the shield 38 is in an extended position and surrounds the needle 35, protecting the needle 35.

As illustrated, the shield 38 is mounted to a recess 39 of the housing 31 on guides 40 that permit the shield 38 to slide into and out of the recess 39. The shield 38 may include engaging members that interact with the guides 40 to permit the sliding movement. Alternatively, the guides 40 may comprise engaging members that interact with the shield 38 to permit the sliding movement. The guides 40 may comprise a groove, a protrusion, a linear bearing, or other feature that permits movement of the shield 38. The guides 40 may be omitted if the shield 38 and recess 39 are shaped correspondingly so that the shield 38 slides into and out of the recess 39.

A biasing member, in this example a spring 44, is arranged to urge the shield 38 into the extended position shown in FIG. 2A.

In this way, when the drug delivery device 30 is placed against a skin 37 of a user for use, as shown in FIG. 2B, the shield 38 can be deflected into the retracted position, within the recess 39, thereby allowing the needle 35 to pierce the user's skin 37. As the drug delivery device 30 is pressed against the skin 37 of the user, the shield 38 slides into the retracted position within the recess 39 and the spring 44 is compressed, while the needle 35 becomes exposed and is pushed into the skin 37 of the user.

The bottom surface 36 of the drug delivery device 30 may have an attachment mechanism, for example an adhesive, to attach the drug delivery device 30 to the skin 37 of the user. Alternatively, a strap may be provided to hold the drug delivery device 30 in place on the skin 37 of the user.

The drug delivery device 30 is in the condition shown in FIG. 2B for the duration of use, that is, until the appropriate amount of medicament has been injected. After use, the drug delivery device 30 is removed from the skin 37 of the user and the spring 44 returns the shield 38 to the extended position, so that the shield 38 and needle 35 are arranged as shown in FIG. 2A after use. Therefore, after use, the shield 38 surrounds the needle 35 and protects the needle 35 and also protects the user and others from being pierced by the needle 35 after use.

In this example, the shield 38 has a generally cylindrical shape and surrounds the needle 35, with the needle 35 being located in the hollow interior of the shield 38. However, in alternative examples the shield 38 may have an alternative tubular shape, for example square, rectangular, or hexagonal with the needle 35 being located within the shield 38. Alternatively, the shield 38 may comprise a wall that abuts against the needle 35 without surrounding the needle 35.

As shown in FIG. 2C, after use, when the shield 38 has returned to the extended position, the shield 38 and the needle 35 can be removed from the housing 31. The shield 38 and the needle 35 together form a needle assembly 41 that can be detached from the housing 31 and disposed of. By detaching the shield 38 and needle 35 together the detached needle assembly 41 does not have any protruding needle 35, making it safer to remove the needle 35 from the housing 31 and also to dispose of it.

The needle 35 may be movably connected to the shield 38. For example, the needle 35 may include a protrusion that is received in a groove within the shield 38. Alternatively, the shield 18 may include a tube in which the needle 15 is received, allowing the needle 15 to slide within the tube. In this way, the shield 38 can move from the extended position to the retracted position while the needle 35 stays stationary (as shown in FIG. 2B). However, once the needle assembly 41 is removed from the housing 31 the protrusion and groove hold the needle 35 and shield 38 together.

As shown in FIG. 2C, on removal of the needle assembly 41 from the housing 31 the pipe 34 is disconnected from the needle 35. To allow this, the pipe 34 is detachably connected to the needle 35. For example, an end 42 of the needle 35 is received in the end 43 of the pipe 34, and the pipe 34 can be pulled off the end 42 of the needle 35. The end 42 of the needle 35 may include a bulbous section that is received in the end 43 of the pipe 34, to increase the holding force between the pipe 34 and the needle 35 but still allowing the pipe 34 to be pulled off the needle 35. Alternatively, the end 23 of the pipe 14 may be received in the end 22 of the needle 15.

As explained above the needle assembly 41, which includes the shield 38 and the needle 35, is detachable from the housing 31.

In one example, the needle assembly 41 may be threadingly attached to the housing 31, with the recess 39 of the housing 31 comprising a female thread and the shield 38 comprising a male thread (or vice versa). In this way, twisting the shield 38 relative to the housing 31 will unscrew the needle assembly 41 and allow the needle assembly 41 to be pulled away from the housing 31, which in turn disconnects the pipe 34 from the needle 35.

In another example, the needle assembly 41 is attached to the housing 31 by a bayonet fitting, allowing the needle assembly 41 to be detached from the housing 31. In this example, the recess 39 of the housing 31 may include one or more lugs that engage with a bayonet slot on the shield 38 (or vice versa).

In another example, the needle assembly 41 is attached to the housing 31 by a combination of a thread attachment and a bayonet attachment.

In another example, the needle assembly 41 is attached to the housing 31 by a push-fit, where the shield 38 is pushed into the recess 39 and held by friction or by some part of the recess 39 and/or shield 38 deforming under pressure. Deformable holding tabs may be provided on the housing 11 and/or shield 18 for this purpose. Such a push-fit allows the needle assembly 41 to be detached from the housing 31 by pulling the shield 38 out of the recess 39.

In an alternative embodiment similar to that illustrated in FIGS. 2A to 2C, the needle 35 is in a fixed extended position relative to the shield 38 and extends past the plane of the bottom surface 36 of the housing 31. In this embodiment, the shield 38 is initially in a retracted position, within the housing 31, and moves from the retracted position to the extended position after the drug delivery device 30 has been used. In this example, the drug delivery device 30 may include a bung or cap for the needle 35 that is removed prior to use. The needle assembly 41 can be detached after use in the same way as the other embodiments.

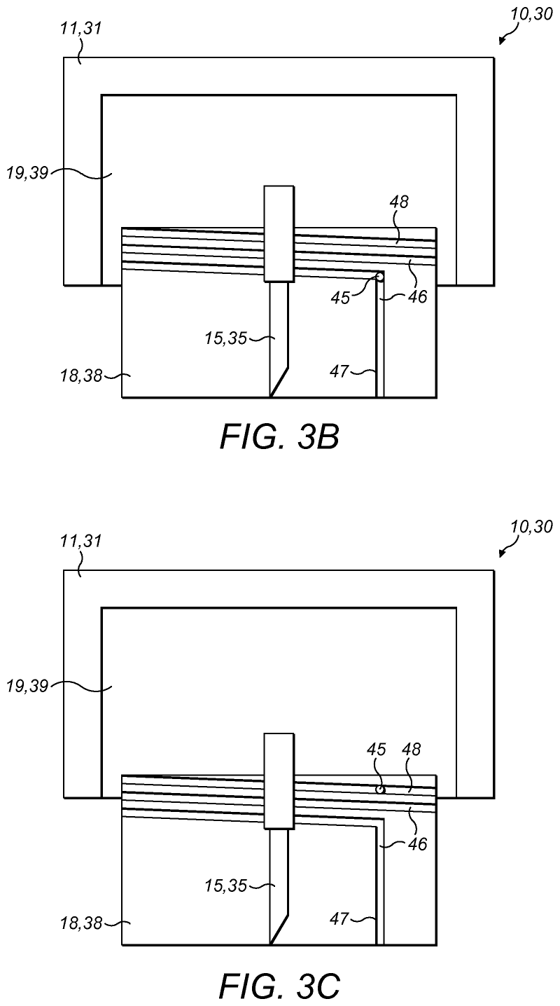

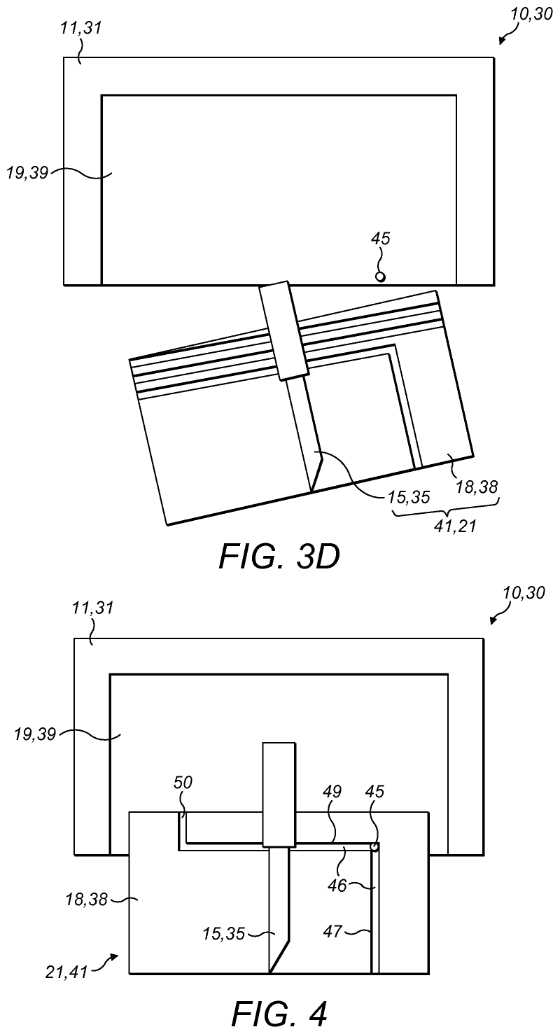

FIGS. 3A, 3B, 3C and 3D illustrate an example of a removable attachment between the needle assembly 21, 41 and the housing 11, 31. The removable attachment may be provided for the example of FIGS. 1A to 1D or the example of FIGS. 2A to 2C.

In the example shown in FIG. 3A, the shield 18, 38 is in a retracted position within the housing 11, 31 and the needle 15, 35 is in an extended position. This is the condition of the shield 18, 38 and needle 15, 35 during use of the drug delivery device 10 described with reference to of FIGS. 1A to 1D and the drug delivery device 30 described with reference to FIGS. 2A to 2C.

Also shown in FIG. 3A, the recess 19, 39 of the housing 11, 31, in which the shield 18, 38 is received, includes a lug 45 that engages with a groove 46 on the shield 18, 38. The groove 46 is on the outer surface of the shield 18, 38. The groove 46 includes a straight section 47 and in the position shown in FIG. 3A the lug 45 is positioned in the straight section 47 of the groove 46. The straight section 47 extends in the same direction as the shield 18, 38 moves between the retracted and extended positions, therefore allowing the shield 18, 38 to move from the retracted position of FIG. 3A to the extended position of FIG. 3B without rotation. As shown in FIG. 3B, in the extended position the lug 45 is now located at an opposite end of the straight section 47 of the groove 46. In alternative examples, the straight section 47 may be angled or even curved, so long as the straight section 47 is arranged such that the shield 18, 38 moves from the retracted position to the extended position as the lug 45 passes along the straight section 47.

As illustrated in FIG. 3C and FIG. 3D, from the extended position the groove 46 also includes a thread section 48 extending from the top of the straight section 47, so that the shield 18, 38 can be rotated to remove it from the housing 11, 31. The thread section 48 extends about the outer surface of the shield 18, 38.

In alternative embodiments the lug 45 can be provided on the shield 18, 38 and the groove 46 can be formed in the recess 19, 39.

In an alternative embodiment, illustrated in FIG. 4, the groove 46 includes a straight section 47, similar to that of FIG. 3A to 3B, that allows the shield 18, 38 to move into the extended position. The groove 46 also includes a bayonet section 49, 50, and the shield 18, 38 can be removed by rotating the shield 18, 38 until the lug 45 reaches the end of the groove 46, which allows the shield 18, 38 to be detached from the housing 11, 31.

In this particular embodiment, the bayonet section 49, 50 of the groove 46 includes a transverse section 49 that extends transverse to the direction of movement of the shield 18, 38 relative to the housing 11, 31, and an exit section 50 that extends from the transverse section 49 to the end of the shield 18, 38. This arrangement allows the needle assembly 21, 41 to be removed by first rotating the shield 18, 38 relative to the housing 11, 31 so that the lug 45 is moved along the transverse section 49, and then pulling the shield 18, 38 away from the housing 11, 31 so that the lug 45 is moved along the exit section 50.

It will be appreciated that the groove 46 may alternatively be formed within the recess 19, 39, and the lug 45 provided on the shield 18, 38.

In an alternative embodiment, the needle assembly 21, 41 (needle 15, 35, and shield 18, 38) can be removed from the housing 11, 31 by pulling the shield 18, 38 away from the housing 11, 31. The shield 18, 38 may include deformable tabs that hold the shield 18, 38 in the recess 19, 39 of the housing 11, 31 until the shield 18, 38 is pulled, at which point the tabs can deform, allowing the shield 18, 38 to be removed. Alternatively, the shield 18, 38 may include breakable tabs that hold the shield 18, 38 in the recess 19, 39 of the housing 11, 31 until the shield 18, 38 is pulled, at which point the tabs are broken, allowing the shield 18, 38 to be removed. Such breakable tabs may also prevent the needle assembly 21, 41 from being replaced in the drug delivery device 10, 30, providing tamper evidence.

As explained previously, the needle 15, 35 and the shield 18, 38 are slidably connected, so that they can independently move between the retracted and the extended position, but when the shield 18, 38 is unscrewed from the housing 11, 31 the needle 15, 35 is also removed. Therefore, in this example, the entire needle assembly 21, 41 (including needle 15, 35 and shield 18, 38) can be unscrewed from the housing 11, 31 and disposed of separately to the remainder of the drug delivery device 10, 30.

Additionally, a replacement needle assembly 21, 41 may be attached to the drug delivery device 10, 30, to allow the remainder of the drug delivery device 10, 30 to be reused.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injector device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injector devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin; B29-N—(N-lithocholyl-gamma-glutamyl)-des(B30) human insulin; B29-N-(ω-carboxyheptadecanoyl)-des(B30) human insulin and B29-N-(ω-carboxyhepta¬decanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

1-15. (canceled)

16. A drug delivery device comprising:
a housing having a surface adapted to be placed against a skin of a user during use of the drug delivery device;
an attachment mechanism for holding the drug delivery device on the skin of the user; and,
a needle assembly having:
a needle that protrudes from the surface for delivery of a drug; and
a shield that surrounds the needle after use of the drug delivery device,
and wherein the needle assembly is detachable from the housing for disposal.

17. The drug delivery device of claim 16, wherein the needle is movable between a retracted position and an extended position in which the needle protrudes from the surface.

18. The drug delivery device of claim 16, wherein the needle assembly and housing are threadingly attached.

19. The drug delivery device of claim 16, further comprising a locking mechanism arranged to lock the needle to the shield.

20. The drug delivery device of claim 16, further comprising a reservoir for holding the drug, and a fluid connector between the reservoir and the needle for carrying the drug from the reservoir to the needle, wherein the fluid connector is flexible or extendable.

21. The drug delivery device of claim 16, further comprising a reservoir for holding the drug, and a fluid connector between the reservoir and the needle for carrying the drug from the reservoir to the needle, wherein the fluid connector is flexible and extendable.

22. The drug delivery device of claim 16, further comprising a reservoir that contains the drug.

23. The drug delivery device of claim 17, wherein the needle is slidably mounted to the shield.

24. The drug delivery device of claim 17, further comprising a needle actuation mechanism adapted to move the needle from the retracted position to the extended position.

25. The drug delivery device of claim 16, wherein the shield is moveable between a retracted position and an extended position.

26. The drug delivery device of claim 25, wherein the shield is adapted to move from the retracted position to the extended position after use of the drug delivery device.

27. The drug delivery device of claim 25, further comprising a biasing member arranged to urge the shield towards the extended position.

28. The drug delivery device of claim 25, further comprising a latch adapted to hold the shield in the retracted position prior to use of the drug delivery device.

29. The drug delivery device of claim 25, wherein the shield comprises a groove and the housing comprises a lug that can move within the groove to control movement of the shield relative to the housing.

30. The drug delivery device of claim 29, wherein the groove comprises a first portion that defines movement of the shield from the retracted position to the extended position; and, a second portion that allows the shield to be detached from the housing.

31. A method of using a drug delivery device, the drug delivery device comprising a housing, and a needle assembly having a needle and a shield, wherein the method comprises:
using an attachment mechanism to hold the drug delivery device on a skin of a user;
delivering a drug to the user via the needle;
moving the shield into a position in which it surrounds the needle after delivering the drug to the user; and
detaching the needle assembly from the housing.

32. The method of claim 31, further comprising unlatching a latch adapted to hold the shield in a retracted position.

33. The method of claim 31, further comprising moving the shield between a retracted position and an extended position.

34. The method of claim 31, further comprising moving the needle between a retracted position and an extended position in which the needle protrudes from a surface.

35. The method of claim 31, further comprising sliding the needle relative to the shield.
